(19) United States
(12) Patent Application Publication
(10) Pub. No.: US 2012/0253319 A1
(43) Pub. Date: Oct. 4, 2012

(54) GUIDEWIRE

(75) Inventor: Kenichi MATSUO, Izumi-shi (JP)

(73) Assignee: ASAHI INTECC CO., LTD., Nagoya-shi (JP)

(21) Appl. No.: 13/420,191

(22) Filed: Mar. 14, 2012

(30) Foreign Application Priority Data
Mar. 31, 2011 (JP) 2011-076947

(51) Int. Cl. A61M 25/09

(52) U.S. Cl. 604/528

(57) ABSTRACT

A guidewire includes a core shaft having a front end portion and a rear end portion, a helical coil in which the front end portion is inserted, and a solder part with which the front end portion and the helical coil are bonded to each other. The helical coil is formed of a wire having a plurality of grooves in a surface of the wire, the grooves being formed such that more grooves are formed along an axial direction of the wire than along a circumferential direction of the wire.

### CROSS-REFERENCE TO RELATED APPLICATIONS

This application claims priority to Japanese Patent Application No. 2011-076947 filed in the Japan Patent Office on Mar. 31, 2011, the entire contents of which are hereby incorporated by reference.

### BACKGROUND

The disclosed embodiments relate to a medical device. More specifically, the disclosed embodiments relate to a guidewire. Guidewires are medical devices used to guide a device such as a balloon or a stent to a lesion in percutaneous transluminal coronary angioplasty (PTCA).

Japanese Unexamined Patent Application Publication No. 2007-135645 discloses an example of such a guidewire which includes a core shaft including a front end portion and a rear end portion, a helical coil that covers the front end portion of the core shaft, a solder part that bonds the core shaft and the helical coil together, and a resin coating layer formed at the surface of the helical coil.

The front end portion and the rear end portion of the core shaft respectively serve as a distal portion and a proximal portion of the guidewire. The distal portion of the guidewire is inserted into the body, and the proximal portion of the guidewire is operated by an operator, such as a doctor.

### SUMMARY

In the guidewire described in Japanese Unexamined Patent Application Publication No. 2007-135645, the helical coil is formed of a wire having a roughened surface so that the resin coating layer tightly adheres to the helical coil.

However, in such a guidewire, the bonding strength between the helical coil and the solder part is not sufficiently high. Therefore, the helical coil is easily detached from the guidewire in response to an external force, such as a pulling force, when the guidewire is being used.

In particular, when the guidewire includes a helical coil formed of a wire including tungsten, it is relatively difficult to bond the helical coil with the solder part. Therefore, the bonding strength between the helical coil and the solder part in such a guidewire is even lower.

As a result of diligent studies conducted by the inventor of the present invention to solve the above-described problem, the present inventor has determined that when a helical coil formed of a wire with a plurality of grooves that extend in a certain direction is used, the helical coil can be prevented from becoming detached from the guidewire. Thus, the guidewire described herein has been produced.

More specifically, a guidewire according to an embodiment of the present invention includes a core shaft having a front end portion and a rear end portion, a helical coil in which the front end portion is inserted, and a solder part with which the front end portion and the helical coil are bonded to each other. The helical coil is formed of a wire having a plurality of grooves in a surface of the wire, the grooves being formed such that more grooves are substantially formed along an axial direction of the wire than along a circumferential direction of the wire.

### BRIEF DESCRIPTION OF THE DRAWINGS

FIG. 1 is a schematic sectional view of a guidewire according to an embodiment of the present invention taken along a longitudinal direction of the guidewire.

FIG. 2A is a schematic perspective view of a helical coil included in the guidewire illustrated in FIG. 1.

FIG. 2B is a schematic perspective view of a wire obtained by stretching the helical coil illustrated in FIG. 2A.

FIG. 2C is a schematic vertical sectional view of the wire illustrated in FIG. 2B taken along a plane perpendicular to the longitudinal direction of the wire.

FIG. 3 is an enlarged sectional view of an area around a rear-end solder part in the guidewire illustrated in FIG. 1.

FIG. 4A is a scanning electron micrograph that shows an enlarged view of a tungsten wire forming a helical coil in a model of the guidewire according to the present invention.

FIG. 4B is a macrophotograph of a front end portion of the model including the tungsten wire illustrated in FIG. 4A.

FIG. 5A is a scanning electron micrograph that shows an enlarged view of a tungsten wire forming a helical coil in a model of a guidewire that was not subjected to the roughening process and that lacks the grooves.

FIG. 5B is a macrophotograph of a front end portion of the model including the tungsten wire illustrated in FIG. 5A.

### DETAILED DESCRIPTION OF EMBODIMENTS

The structure of the guidewire according to an embodiment of the present invention and effects of the guidewire will now be described in detail.

In the following description, a distal portion of a guidewire and a front end portion of a core shaft are denoted by the same reference numeral, and a proximal portion of the guidewire and a rear end portion of the core shaft are denoted by the same reference numeral. In addition, a portion of the helical coil may be shown by broken lines, and detailed illustration of the portion of the helical coil may thus be omitted.

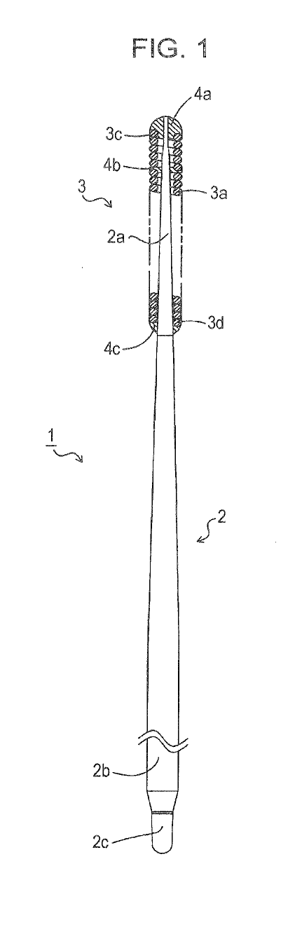

A guidewire 1 according to an embodiment of the present invention illustrated in FIG. 1 includes a core shaft 2 having a front end portion 2a and a rear end portion 2b, a helical coil 3 in which the front end portion 2a is inserted, and solder parts (4a, 4b, and 4c) that bond the front end portion 2a and the helical coil 3 to each other. In the example illustrated in FIG. 1, the solder parts include a front-end solder part 4a, an intermediate solder part 4b, and a rear-end solder part 4c. However, the number of solder parts is not limited to three. For example, the solder parts may include only the front-end solder part and the rear-end solder part. Alternatively, two or more intermediate solder parts may be provided.

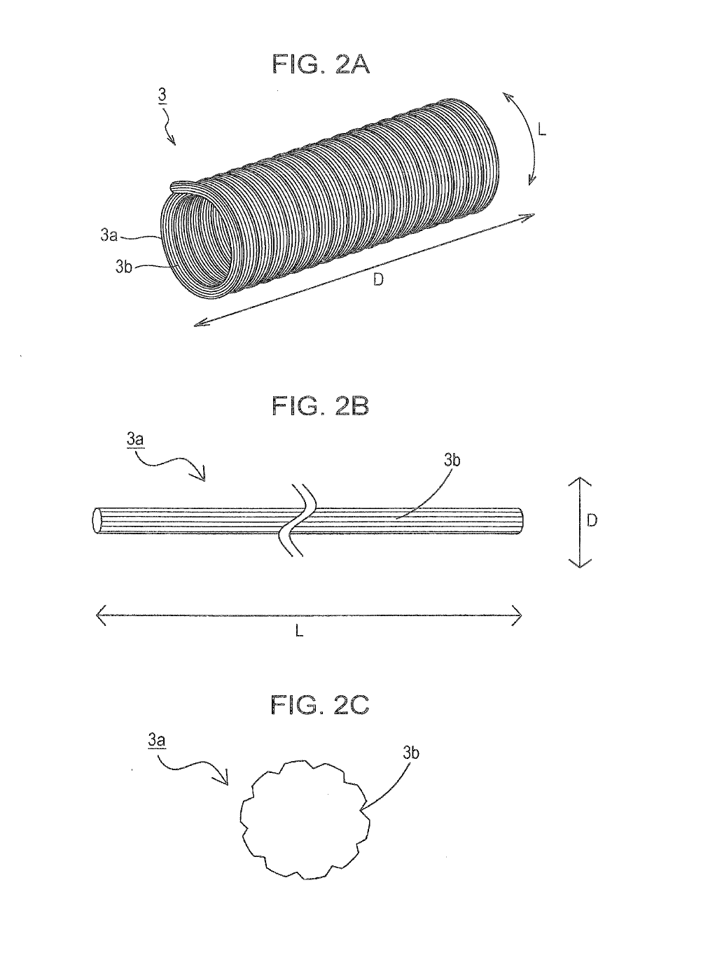

As illustrated in FIGS. 2A, 2B, and 2C, a plurality of grooves 3b are formed in the surface of a wire 3a which forms the helical coil 3. In the embodiment shown in FIGS. 2A, 2B, and 2C, the plurality of grooves includes substantially only grooves formed along the axial direction of the wire. However, the plurality of grooves formed in the surface of the guidewire may further include (not shown) grooves formed along the circumferential direction. In this case, more grooves 3b are formed along an axial direction L of the wire 3a than along a circumferential direction D of the wire 3a.

In this specification, when an imaginary straight line is drawn along the axial direction of the wire that forms the helical coil, grooves that are parallel to the imaginary straight line or grooves that are inclined in directions close to the imaginary straight line are called the grooves formed along the axial direction of the wire. In addition, when an imaginary perpendicular line that is perpendicular to the imaginary straight line is drawn, grooves that are parallel to the imaginary perpendicular line or grooves that are inclined in directions close to the imaginary perpendicular line are called the grooves formed along the circumferential direction of the wire. In addition, the state in which more grooves are formed along the axial direction of the wire than along the circumferential direction of the wire is the state in which the number of grooves formed along the axial direction of the wire is larger than the number of grooves formed along the circumferential direction of the wire, or the state in which grooves formed along the axial direction of the wire are substantially only formed or visible on the surface of the wire.

The directions along which the grooves are formed and the number of grooves can be determined by visual observation or observation using an optical microscope or a scanning electron microscope.

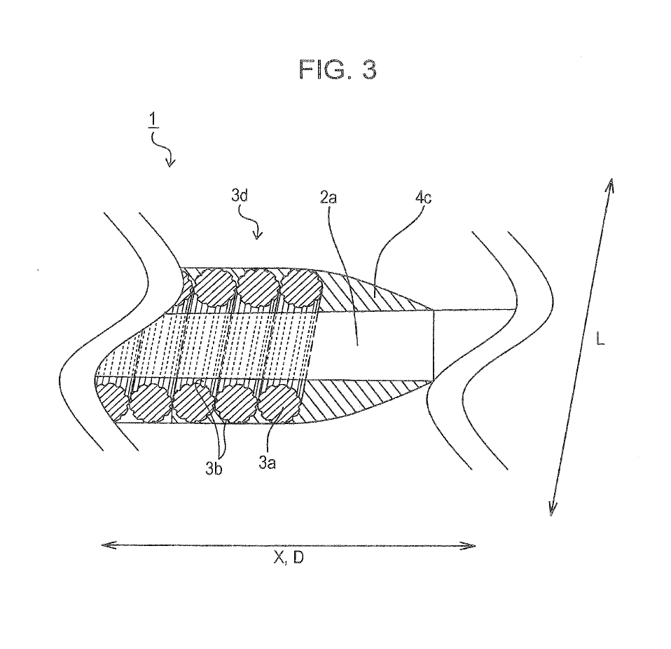

In the guidewire 1 according to an embodiment of the present invention in which the helical coil 3 having the above-described structure and the front end portion 2a of the core shaft 2 are bonded to each other by the solder parts 4a, 4b, and 4c, the grooves 3b in the helical coil 3 are filled with solder that forms the solder parts 4a, 4h, and 4c. Therefore, the helical coil 3 is strongly bonded with the solder parts 4a, 4b, and 4c as illustrated in, for example, FIG. 3, which is the enlarged view of the area around the rear-end solder part 4c.

In addition, as illustrated in FIG. 3, a longitudinal direction X of the guidewire 1 and the axial direction L of the wire 3a, along which the grooves 3b are mainly formed, cross at an angle close to about 90°. Therefore, even when an external force is applied in the longitudinal direction X of the guidewire 1, an anchoring effect against the external force is provided by the grooves formed along the direction substantially perpendicular to the direction in which the external force is applied and the solder with which the grooves are filled. Although the rear-end solder part 4c is explained above as an example, the helical coil 3 is also strongly bonded to the other solder parts, such as the front-end solder part 4a and the intermediate solder part 4b. Also at the other solder parts, the anchoring effect is provided by the grooves 3b and the solder with which the grooves 3b are filled.

With a guidewire according to the related art, when, for example, an operator pulls the guidewire to release the distal portion of the guidewire that has been caught by a lesion, the pulling force is applied mainly in the longitudinal direction of the guidewire. In such a case in the related art, the helical coil is easily detached from the guidewire.

In contrast, the helical coil 3 in the guidewire 1 according to an embodiment of the present invention is strongly bonded to the solder parts 4a, 4b, and 4c, owing to the grooves 3b formed mainly along a certain direction. Since the grooves 3b and the solder with which the grooves 3b are filled provide the anchoring effect, the helical coil 3 is not easily detached from the guidewire 1 even when a pulling force is applied to the guidewire 1 in the longitudinal direction X thereof.

The structure of each component of the guidewire 1 according to the present embodiment will now be described.

The rear end portion 2b illustrated in FIG. 1 is tapered such that the diameter thereof decreases toward the front end of the core shaft 2. A connector portion 2c that provides connection to an extension guidewire or the like is provided at the rear end of the rear end portion 2b. In an alternate embodiment, the rear end portion 2b may instead have a cylindrical shape with a substantially constant diameter.

The front end of the rear end portion 2b is coupled to the front end portion 2a, which is tapered such that the diameter thereof decreases toward the front end of the core shaft 2.

Thus, the diameter of the core shaft 2 decreases toward the front end portion 2a from the rear end portion 2b. Therefore, the flexibility of the core shaft 2 increases toward the front end portion 2a from the rear end portion 2b.

The material of the core shaft 2 may be, for example, a stainless steel, a superelastic alloy such as Ni—Ti alloy, a piano wire, or a tungsten wire. The stainless steel may be, for example, a martensitic stainless steel, a ferritic stainless steel, an austenitic stainless steel, an austenitic-ferritic duplex stainless steel, or a precipitation hardened stainless steel. The material of the core shaft 2 is preferably a stainless steel, and more preferably, an austenitic stainless steel. In particular, SUS304, SUS316, or SUS316L is preferably used.

The helical coil 3 illustrated in FIG. 2A is formed into a tubular shape with a through hole at the center by helically winding a single wire 3a or a plurality of wires 3a.

The plurality of grooves 3b are formed in substantially the entire surface of each wire 3a that forms the helical coil 3. More grooves 3b are formed along the axial direction L of the wire 3a than along the circumferential direction D of the wire 3a.

The grooves 3b may have a maximum depth of, for example, 1.0 to 100 μm, and a length of, for example, 0.1 to 10 mm.

The material of the wire 3a may be, for example, a stainless steel such as a martensitic stainless steel, a ferritic stainless steel, an austenitic stainless steel, an austenitic-ferritic duplex stainless steel, or a precipitation hardened stainless steel, a superelastic alloy such as a Ni—Ti alloy, an X-ray impermeable metal such as platinum, gold, tungsten, tantalum, or iridium, or an alloy of the X ray impermeable metals. In particular, tungsten or a tungsten alloy is preferably used.

As illustrated in FIG. 1, the front end portion 2a is inserted in the helical coil 3 so that the helical coil 3 covers the front end portion 2a. The front end portion 2a is spaced by a predetermined distance from the inner wall of the helical coil 3.

The adjacent portions of the wire 3a are in contact with each other over the entire area of the helical coil 3 from a front end 3c to a rear end 3d. Therefore, the helical coil 3 has a substantially constant flexural property over the entire body thereof. In the area around the front end of the helical coil, pitches may be formed between the adjacent portions of the wire. In such a case, the flexibility of the helical coil is increased at the area around the front end thereof. In the area around the rear end of the helical coil, the wire may be densely wound such that the adjacent portions of the wire are in contact with each other. In such a case, the helical coil is not easily twisted even when a twisting force is applied to the helical coil in the area around the rear end thereof. As a result, a torque generated when the proximal portion of the guidewire is rotated can be efficiently transmitted to the distal portion of the guidewire.

The front end 3c of the helical coil 3 and the front end of the front end portion 2a are fixed to each other with the front-end solder part 4a, which has a hemispherical shape. The helical coil 3 and the front end portion 2a are fixed to each other with the intermediate solder part 4b at an intermediate position that is separated from the front end 3c toward the rear end 3d of the helical coil 3. The rear end 3d of the helical coil 3 and the rear end of the front end portion 2a are fixed to each other with the rear-end solder part 4c.

The material of the solder that forms the front-end solder part 4a, the intermediate solder part 4b, and the rear-end solder part 4c may be, for example, an aluminum alloy, Ag, Au, Zn, Sn—Pb alloy, Sn—Au alloy, Pb—Ag alloy, or Sn—Ag alloy. In particular, Au, Sn—Au alloy, or Sn—Ag alloy is preferably used. This is because these materials achieve high bonding strength at the solder parts.

The guidewire according to the present embodiment may be manufactured by the following method.

(1) The core shaft is formed by forming a wire rod into the above-described shape by, for example, a taper cutting process or a pressing process.

(2) A wire for forming the helical coil is subjected to a roughening process. The roughening process may be performed by, for example, immersing the wire in a high-temperature alkaline solution, electropolishing the wire in an alkaline solution, or polishing the wire by reciprocating an abrasive paper with a certain grit size in the axial direction of the wire.

The estimated mechanisms of immersing the wire in a high-temperature alkaline solution and electropolishing are described below. The tungsten wire is formed by drawing of a thick tungsten wire, and therefore tungsten crystal grains contained in the wire are seemed to be likely to align on the axial direction of the wire. As a result, the grooves which are formed by solving the grains in the immersing or electropolishing process may contain more grooves formed along the axial direction and fewer grooves formed along the circumferential direction.

Selecting an appropriate roughening process allows to form only grooves which extend along the axial direction, or to form both grooves which extend along the axial direction and circumferential direction of the wire with the number of grooves formed in the axial direction being more than the number of grooves formed in the circumferential direction.

(3) The helical coil is formed by helically winding one or more wires that have been subjected to the roughening process.

(4) The front end portion of the core shaft is inserted into the helical coil, and the core shaft and the helical coil are soldered to each other at predetermined positions. Thus, the guidewire of the present embodiment is manufactured.

The effects of the guidewire according to the present embodiment will now be described.

(1) In the guidewire according to the present embodiment, a plurality of grooves are formed in the surface of the wire that forms the helical coil such that more grooves are formed along the axial direction of the wire than along the circumferential direction of the wire. Therefore, the helical coil is strongly bonded with the solder parts, and an anchoring effect can be obtained by the grooves and the solder with which the grooves are filled. Therefore, the helical coil is not easily detached from the guidewire even when a pulling force is applied to the guidewire in the longitudinal direction thereof.

(2) Even when the wire that forms the helical coil includes tungsten, which has a relatively low wettability to solder, the grooves can be filled with the solder and the helical coil can be strongly bonded with the solder parts. In particular, when the wire includes tungsten and the solder is Sn—Ag alloy, the above-described effects can be usefully exploited. In contrast, when the wire including tungsten does not have the above-described grooves, the wire and the solder repel each other and the helical coil cannot be easily bonded with the solder parts. As a result, the bonding strength is reduced.

(3) When the wire that forms the helical coil includes tungsten, which is relatively strong among the X-ray impermeable metals, the occurrence of breakage can be reduced. In addition, an inexpensive helical coil can be produced with low environmental load.

As a model of the guidewire according to the present embodiment, Model 1 was manufactured by the following process.

That is, a stainless steel rod having the shape of the core shaft, a helical coil (tungsten coil), and Sn—Ag alloy solder were prepared. The stainless steel rod had a diameter of 0.081 mm and a length of 200 mm. The helical coil (tungsten coil) had an outer diameter of 0.342 mm, an inner diameter of 0.180 mm, and a length of 120 mm, and was formed by helically winding a tungsten wire with a diameter of 0.081 mm.

The tungsten wire was subjected to a roughening process (NaOH aqueous solution, 600 degrees, 20 seconds) in advance.

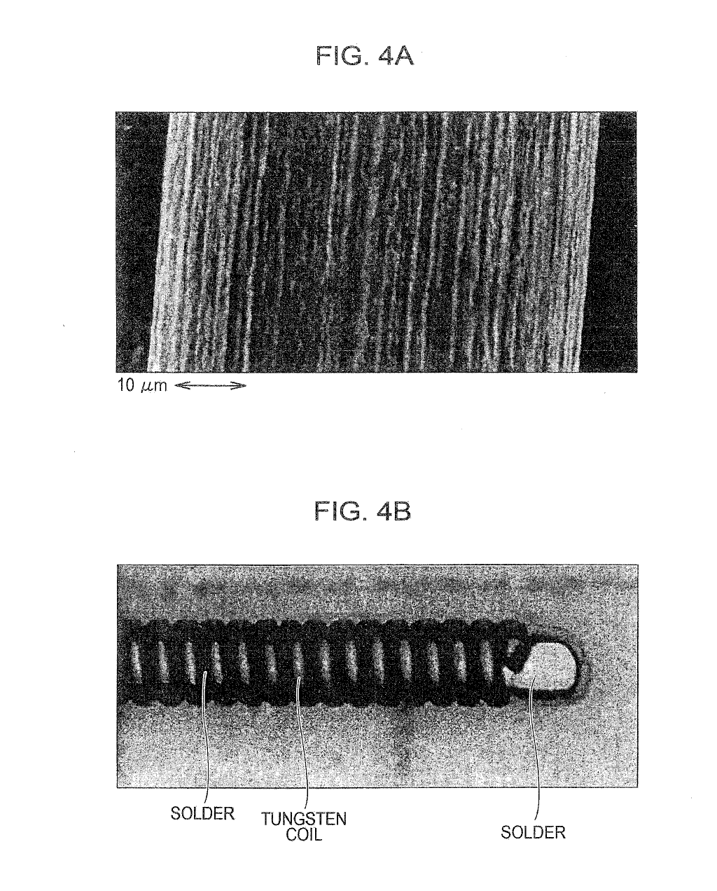

The roughened surface of the tungsten wire was observed with a scanning electron microscope at a magnification of 1,800. FIG. 4A is a scanning electron micrograph that shows an enlarged view of the tungsten wire forming the helical coil in the model of the guidewire according to an embodiment of the present invention.

As is clear from FIG. 4A, a plurality of grooves extending along the axial direction (vertical direction in FIG. 4A) of the tungsten wire were formed in the surface of the tungsten wire after the roughening process. Any grooves formed along the circumferential direction (horizontal direction in FIG. 4A) of the wire are substantially not visible in FIG. 4A. However, more grooves were formed along the axial direction (vertical direction in FIG. 4A) of the wire than along the circumferential direction (horizontal direction in FIG. 4A) of the wire.

The manufacturing process of Model I will be further explained. The prepared stainless steel rod was inserted into the helical coil. Then, the stainless steel rod and the helical coil were bonded together with the Sn—Ag alloy solder. Model 1 was manufactured by the above-described process.

The state of the solder parts of Model 1 was observed. FIG. 4B is a macrophotograph of the front end portion of Model 1 including the tungsten wire illustrated in FIG. 4A.

As illustrated in FIG. 4B, in Model 1, spaces between the adjacent portions of the wire in the helical coil (tungsten coil) were filled with the solder, and the solder did not protrude from the helical coil. Thus, it was confirmed that the helical coil was reliably bonded with the solder parts. Therefore, the bonding strength between the helical coil and the solder parts is high.

Model 2 was manufactured by a process similar to that of Model 1 except the roughening process was not performed.

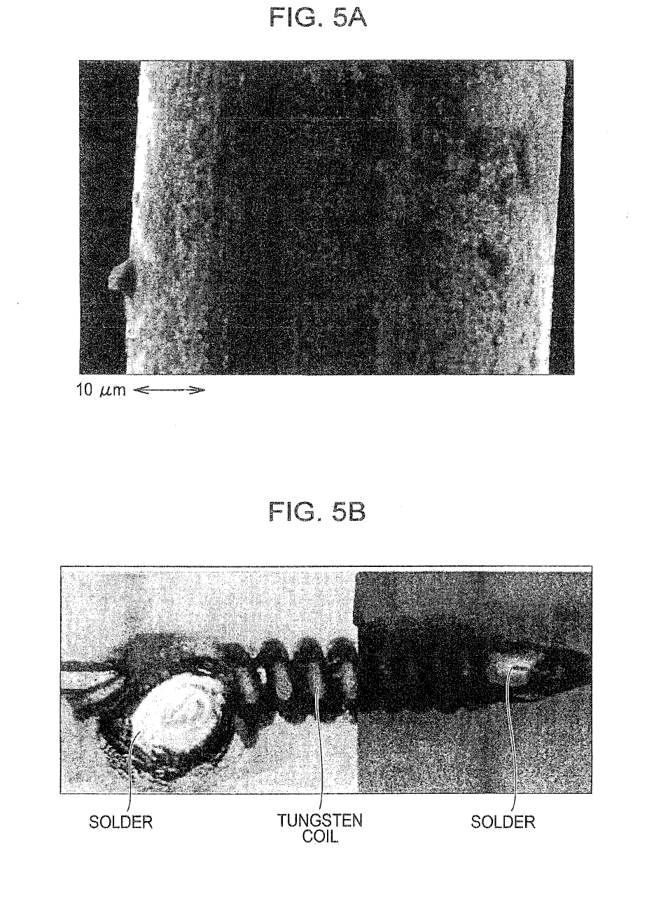

The surface of the tungsten wire that was not subjected to the roughening process was observed with a scanning electron microscope at a magnification of 1,800. FIG. 5A is a scanning electron micrograph that shows an enlarged view of the tungsten wire forming the helical coil in the model of the guidewire that was not subjected to the roughening process and that lacks the grooves.

As is clear from FIG. 5A, the grooves are not formed in the surface of the tungsten wire that was not subjected to the roughening process.

The state of the solder parts of Model 2 was observed. FIG. 5B is a macrophotograph of the front end portion of Model 2 including the tungsten wire illustrated in FIG. 5A. In FIG. 5B, an intermediate portion of the helical coil is omitted and only the areas around the front and rear ends of the helical coil are shown.

As illustrated in FIG. 5B, in Model 2, spaces between the adjacent portions of the wire in the helical coil are not filled with the solder, and the solder largely protrudes from the helical coil. Therefore, the bonding strength between the helical coil and the solder parts is low.

In the guidewire according to an embodiment of the present invention, more grooves are formed along the axial direction of the wire than along the circumferential direction of the wire, and the grooves may be provided over the entire area of the surface of the wire. Alternatively, the grooves may be provided only in the areas in which the helical coil is bonded with the solder parts.

In the guidewire according to an embodiment of the present invention, the helical coil may be tapered such that the diameter thereof decreases toward the front end from the rear end of the helical coil. The guidewire including a helical coil having such a shape is desirable since the guidewire can be easily inserted into a hard lesion, such as a chronic total occlusion lesion.

The outer surface of the guidewire according to an embodiment of the present invention may be covered with a hydrophilic material. In such a case, the guidewire can be smoothly moved through a guiding catheter, a tube, or a body tissue by reducing the sliding friction.

Examples of the hydrophilic material include cellulose-based high-polymer materials, polyethylene-oxide-based high-polymer materials, maleic-anhydride-based high-polymer materials (e.g., maleic anhydride copolymers such as methyl vinyl ether-maleic anhydride copolymers), acrylamide-based high-polymer materials (e.g., polyacrylamides and polyglycidyl methacrylate-dimethylacrylamide (PGMA-DMAA) block copolymers), water-soluble nylons, polyvinyl alcohols, polyvinyl pyrrolidones, and hyaluronates. In particular, hyaluronates are preferably used.

While the foregoing embodiments have been shown and described in detail, the foregoing description is in all aspects illustrative and not restrictive. It is therefore understood that numerous modifications and variations can be devised without departing from the spirit and scope of the invention.

What is claimed is:

1. A guidewire comprising:
a core shaft having a front end portion and a rear end portion;
a helical coil in which the front end portion is inserted; and
a solder part with which the front end portion and the helical coil are bonded to each other,
wherein the helical coil includes a wire having a plurality of grooves in a surface of the wire, the grooves being formed such that more grooves are substantially formed along an axial direction of the wire than along a circumferential direction of the wire.

2. The guidewire according to claim 1, wherein the wire comprises tungsten.

3. The guidewire according to claim 1, wherein the grooves are substantially filled with solder.

4. The guidewire according to claim 1, wherein the grooves are substantially perpendicular to a longitudinal direction of the guidewire.

5. The guidewire according to claim 1, wherein the core shaft has a substantially tapered shape, such that a diameter of the core shaft decreases toward the front end of the core shaft.

6. The guidewire according to claim 1, wherein a material of the core shaft is selected from the group consisting of a stainless steel, a superelastic alloy, a piano wire and a tungsten wire.

7. The guidewire according to claim 6, wherein the stainless steel can be selected from the group consisting of a martensitic stainless steel, a ferritic stainless steel, an austenitic stainless steel, an austenitic-ferritic duplex stainless steel and a precipitation hardened stainless steel.

8. The guidewire according to claim 1, wherein the grooves have a depth ranging from about 1.0 μm to about 100 μm.

9. The guidewire according to claim 1, wherein the grooves are formed substantially along the entire surface the wire.

10. The guidewire according to claim 1, wherein the grooves have a length ranging from about 0.1 mm to about 10 mm.

11. The guidewire according to claim 1, wherein a material of the wire is selected from the group consisting of a stainless steel, a superelastic alloy, an X-ray impermeable metal and an alloy of X-ray impermeable metals.

12. The guidewire according to claim 1, wherein the helical coil includes pitches that are formed at a front end of the helical coil.

13. The guidewire according to claim 1, wherein the solder part includes a front end solder part having a hemispherical shape, the front end solder part fixing the helical coil to the core shaft

14. The guidewire according to claim 1, wherein a material of the solder part is selected from the group consisting of an aluminum alloy, an Sn—Pb alloy, an Sn—Au alloy, an Pb—Ag alloy and Sn—Ag alloy.

15. The guidewire according to claim 3, wherein the solder does not protrude from the helical coil.

16. The guidewire according to claim 1, wherein the grooves are provided only in areas in which the helical coil is bonded with the solder part.
